(19) United States
(12) Patent Application Publication
(10) Pub. No.: US 2021/0290804 A1
(43) Pub. Date: Sep. 23, 2021

(54) Disinfection Methods Using Concentrated Gaseous Ozone

(71) Applicant: WillowPure, LLC, Denver, CO (US)

(72) Inventors: Jill Lynn Ellsworth, Aurora, CO (US); Jason Robert Ellsworth, Aurora, CO (US)

(21) Appl. No.: 17/206,311

(22) Filed: Mar. 19, 2021

Related U.S. Application Data
(60) Provisional application No. 62/992,024, filed on Mar. 19, 2020.

(51) Int. Cl. A61L 2/20

(52) U.S. Cl. CPC A61L 2/202; A61L 2202/14; A61L 2202/24; A61L 2202/26

(57) ABSTRACT

Disinfection methods using concentrated gaseous ozone. The methods are useful for disinfecting virus-contaminated items of personal protective equipment (PPE) and paper currency.

### CROSS-REFERENCE TO RELATED APPLICATIONS

This application claims benefit of U.S. Provisional Application No. 62/992,024, filed on Mar. 19, 2020, the entire contents of which are specifically incorporated by reference herein.

### FIELD OF THE DISCLOSURE

This disclosure relates to disinfection methods using concentrated gaseous ozone.

### BACKGROUND

Viruses such as SARS-CoV-2 can contaminate items of personal protective equipment (PPE) used by health care professionals and others. Facilities at particular risk of such contamination include hospitals, emergency rooms, nursing homes and other health care settings. It would be advantageous to decontaminate an item of PPE after its use so that it could be used again, rather than discarding it. This in turn may avoid or lessen the impact of shortages in availability of the equipment. Some facilities may also prefer to recycle equipment even in the absence of any shortages.

Viruses can also reside on paper currency, creating a risk of spread through distribution of the money in commerce. It would be advantageous to decontaminate paper currency to reduce this risk of virus transmission.

### SUMMARY

Embodiments of the disclosure include methods for disinfecting virus-contaminated items of personal protective equipment (PPE) and paper currency. The methods include placing the items of PPE or the paper currency in an ozone chamber and exposing them to concentrated ozone gas.

Treatment of a previously used and contaminated item of PPE according to the disclosure can render the item safer for re-use in the event of an equipment shortage. Treating paper currency according to the disclosure can make it safer for distribution and personal contact.

### BRIEF DESCRIPTION OF THE DRAWINGS

The accompanying figures constitute a part of this disclosure. The figures serve to provide a further understanding of certain exemplary embodiments. The disclosure and claims are not limited to embodiments illustrated in the figures.

FIG. 1A is an SEM image of a control sample of N95 filter material.

FIG. 1B is an SEM image of a sample of N95 filter material after its exposure to ozone gas as discussed in Example 1.

FIG. 2A illustrates a side view of the placement of spore strips within a stack of bills of paper currency as discussed in Example 3.

FIG. 2B is a perspective view of the stack of bills of paper currency with spore strips inserted in the stack.

### DETAILED DESCRIPTION

Various additional embodiments of the disclosure will now be explained in greater detail. Both the foregoing general description and the following detailed description are exemplary and explanatory only and are not restrictive of this disclosure or of the claims. Any discussion of certain embodiments or features, including those depicted in the figures, serve to illustrate certain exemplary aspects of the disclosure. The disclosure and claims are not limited to the embodiments specifically discussed herein.

One embodiment of the disclosure is a method for disinfecting an item of personal protective equipment (PPE) contaminated with a virus, wherein the item of PPE is a respiratory protective device or a containment gown, which comprises:

placing the item of PPE in an ozone chamber; and

exposing the item of PPE to gaseous ozone in the ozone chamber at a concentration of at least 50 ppm for at least 10 minutes.

The term “disinfecting” means reducing the amount of the virus on the item of PPE. This includes any extent of virus reduction and does not require eliminating the virus completely. “Disinfecting” includes reducing the amount of virus from a level that is harmful to a human to a lower level that is less harmful, or not harmful, to a human. “Disinfecting” also includes reducing the amount of virus from a measurable level to a level that is undetectable. The amount of virus can be quantified using any appropriate techniques, such as a plaque-based assay, a protein-based virus quantification assay, a virus counter, and techniques based on detection and quantification by fluorescence. Reducing the amount of the virus includes reducing the concentration of a virus within a particular surface area or defined volume of an item of PPE. The term “disinfecting” discussed here applies as well to other embodiments of the disclosure directed to disinfecting paper currency.

An item of PPE “contaminated with a virus” means that a virus resides on or within the item of PPE or paper currency, possibly including within the porosity of the item or currency if at least a portion of the item of PPE or currency is porous. In either instance, the virus can be in any physical form, such as in the form of a particle or contained within a droplet such as a respiratory droplet.

The item of PPE is a respiratory protective device or a containment gown. In some embodiments, the item of PPE is a respiratory protective device, such as an N95 mask or a surgical mask. The N95 mask filters out at least 95% of airborne particles having a size of 0.3 microns or more. The “N” designation means the filter is not resistant to oil.

In other embodiments the item of PPE is a containment gown, also knows as an isolation gown. Containment gowns are used to protect the wearer from the spread of infection or illness if the wearer encounters potentially infectious material. Many containment gowns are made of hospital-grade polymer-based products that are believed to be particularly compatible with ozone exposure. In some embodiments, the containment gown is made of a material that comprises polyethylene, polypropylene, or both. In further embodiments, the containment gown is made of a material that comprises polyester, cotton, or both.

Another embodiment of the disclosure is method for disinfecting paper currency contaminated with a virus, which comprises:

placing the paper currency in an ozone chamber; and

exposing the paper currency to gaseous ozone in the ozone chamber at a concentration of at least 50 ppm for at least 10 minutes.

In some embodiments, the paper currency is in a stack of at least 10, at least 50, or at least 100 or more individual pieces of paper currency.

The virus in any embodiment of the disclosure includes any human or animal virus. The virus can be, for example, a respiratory virus or influenza. In some embodiments, the virus is the SARS (severe acute respiratory syndrome) virus (SARS-CoV-1), the virus causing MERS (Middle East respiratory syndrome, MERS-CoV), the virus causing COVID-19 (SARS-CoV-2), or any other coronavirus. Reference to “a” virus herein includes “one or more” viruses.

The ozone chamber can be any appropriate shape or size having an interior volume in which the item of PPE or paper currency can be placed and exposed to the concentrated ozone. Possible materials of construction for the ozone chamber include, for example, plastic, aluminum, an aluminum alloy, anodized aluminum or anodized aluminum alloy, and stainless steel. The ozone chamber may include within its interior volume any trays, hooks, rods, racks, shelves or other accessories to accommodate the placing, hanging or other storage of the PPE or paper currency to be treated.

The ozone chamber may be present as a component of an overall ozone treatment system. Such a system can include, for example, an oxygen concentrator configured to concentrate oxygen from ambient air and an ozone generator configured to establish and adjust the concentration of ozone in the chamber. The system could further comprise processors and memory (for example within a controller) to establish a pre-set concentration and treatment time and automatically make adjustments to maintain the desired conditions in the ozone chamber. Exemplary ozone treatment systems include those described in U.S. Pat. Nos. 10,757,944 and 10,757,945, the entire contents of both of which are specifically incorporated by reference herein. Commercial ozone treatment systems include the WillowPure 5, WillowPure 20 and WillowPure 40 systems from Willow Industries, Inc.

The ozone treatment system may further include safety mechanisms, including but not limited to a destructor for venting gaseous ozone, a mechanism for immediately degrading ozone back to oxygen gas, and a leak sensor in communicative contact with an alarm display and a safety interlock.

Ozone occurs naturally at low levels but can be produced in high concentrations using any of a variety of techniques, such as the corona discharge method, narrow-band UV light method, the cold plasma method, and electrolytic ozone generation. The ozone gas may be included within a feed gas to the ozone chamber that comprises additional components such as oxygen, nitrogen, water vapor, argon and/or carbon dioxide.

Ozone gas is convenient to use for disinfection because it can be generated from air, it readily decomposes to oxygen gas and it does not leave a residue or byproduct on the treated material. In contrast, liquid disinfectants such as bleach, phenol, and formaldehyde can leave toxic residuals behind on surfaces. Ozone gas can also advantageously penetrate the micro-pores of an N95 respiratory protective device, for example, to achieve a significant level of disinfection.

Ozone gas has been used as a surface disinfectant due to its highly oxidative properties. In general, it has been used to inactivate a wide variety of viruses on objects with varying types of surfaces and in aerosols. Hudson et al., “Development of a Practical Method for Using Ozone Gas as a Virus Decontaminating Agent,” Ozone: Science & Engineering, vol 31, pp. 216-223 (2009). The effects of ozone concentration, contact time, different capsid architecture of viruses, and relative humidity on viruses have also been evaluated to conclude that ozone gas treatment can be effective for reducing the viral number. Tseng et al., “Inactivation of Surface Viruses by Gaseous Ozone,” Journal of Environmental Health, vol. 70, pp. 56-62 (2008).

The extent of disinfection of a virus-contaminated material can be approximated by conducting the ozone treatment on bacteria spore strips as bioindicators, such as spore strips containing the Geobacillus or Bacillus species or others. The extent of disinfection achieved on these resistant microorganisms should indicate that at least the same or an even greater extent of disinfection could be achieved on a virus using the same ozone treatment. Examples 2 and 3 of the disclosure illustrate the use of spore strips as bioindicators.

The item of PPE, or the paper currency, can be exposed to gaseous ozone in the ozone chamber at a concentration of at least 50 ppm for at least 10 minutes. The concentration of ozone to which the PPE or currency is exposed may be directly measured within the interior of the ozone chamber or may be estimated based on process inputs and outputs. For example, the concentration of ozone in the chamber may be estimated based on a measured concentration of ozone introduced into an inlet of the chamber (or at a location otherwise upstream of the chamber) or on a measured concentration of ozone at an outlet of the chamber (or at a location otherwise downstream of the chamber).

The ozone concentration values herein should be interpreted as modified by the term “about” whether specifically indicated or not. This is to account for inherent variability around a particular desired concentration target achieved by commercially available ozone generators. For example, when a desired ozone concentration is at least 100 ppm, the actual concentration in an ozone chamber could at least transiently be 95 ppm during operation. For example, reference to a particular ozone ppm value herein includes that exact specified value and values of up to 5 ppm above or below. The exact specified concentration levels, unmodified by the term “about,” also form embodiments of the disclosure.

In an embodiment, the concentration of ozone to which the item of PPE or the paper currency is exposed is between about 1 ppm and about 1000 ppm, about 1 ppm and about 800 ppm, about 1 ppm and about 600 ppm, about 50 ppm and about 400 ppm, about 50 ppm and about 300 ppm, about 100 ppm and about 300 ppm, about 150 ppm and about 250 ppm, or about 180 ppm and about 220 ppm. In some embodiments, the concentration of ozone is greater than 20 ppm, greater than 50 ppm, greater than 75 ppm, greater than 100 ppm, greater than 125 ppm, greater than 150 ppm, greater than 175 ppm, greater than 200 ppm, greater than 225 ppm, greater than 250 ppm, greater than 275 ppm, greater than 300 ppm, greater than 400 ppm, greater than 500 ppm, or greater than 600 ppm. In some embodiments, the concentration of ozone is less than 700 ppm, less than 600 ppm, less than 500 ppm, less than 400 ppm, less than 350 ppm, less than 300 ppm, less than 275 ppm, less than 250 ppm, less than 225 ppm, less than 200 ppm, less than 175 ppm, less than 150 ppm, less than 125 ppm or less than 100 ppm. In another embodiment, the concentration of ozone is between about 190 ppm to about 210 ppm.

The concentration of ozone used can be related to the exposure time necessary to achieve a desired extent of disinfection. For example, a relatively low ozone concentration can be effective at disinfecting a material if used over a longer period of time. If a material is instead subjected to a relatively higher concentration of ozone, then less exposure time is likely needed to disinfect the material.

In an embodiment, the time an item of PPE or the paper currency is exposed to ozone is about 1 minute to about 48 hours, about 2 minutes to about 24 hours, about 3 minutes to about 18 hours, about 4 minutes to about 12 hours, about 5 minutes to about 6 hours, about 6 minutes to about 4 hours, about 7 minutes to about 2 hours, about 8 minutes to about 1.5 hours, about 10 minutes to about 1 hour, about 10 minutes to about 1 hour, about 12 minutes to about 50 minutes, about 14 minutes to about 30 minutes, about 16 minutes to about 25 minutes. In some embodiments, the exposure time is greater than 1 minute, greater than 5 minutes, greater than 10 minutes, greater than 15 minutes, greater than 20 minutes, greater than 30 minutes, greater than 45 minutes, greater than 1 hour, greater than 2 hours, greater than 6 hours, greater than 12 hours, greater than 24 hours or greater than 48 hours. In some embodiments, the exposure time is less than 48 hours, less than 24 hours, less than 12 hours, less than 10 hours, less than 8 hours, less than 6 hours, less than 4 hours, less than 3 hours, less than 2 hours, less than 1.5 hours, less than 1 hour, less than 50 minutes, less than 40 minutes, less than 30 minutes, less than 25 minutes, less than 20 minutes, less than 15 minutes, less than 10 minutes, less than 5 minutes, or less than 2 minutes. In one embodiment, the exposure time is about 20 minutes to about 60 minutes.

### Example 1: Respiratory Protective Device

Processing of N95 mask material. Coupons having a dimension of 0.25 in² (0.5″×0.5″) were cut from a Haylard level 3 N95 medical face mask (Haylard, Alpharetta, Ga.). The coupons were loaded into a WillowPure 20 gaseous ozone disinfection chamber and exposed to ozone at a concentration of 430 ppm for 2.5 hours. The processed coupons were removed from the chamber and delivered to a laboratory for SEM imaging. Unprocessed (control) coupons and the processed coupons were imaged at magnifications of 200× and 500×.

Analysis of N95 mask material. The original N95 filter material consists of woven fibers, with most fibers ranging in diameter from 1 μm to 10 μm. Fiber diameters were used as the initial metric in determining whether the ozone exposure degraded the N95 filter material. 100 random fibers were selected from the SEM images of both the unprocessed coupons and the processed coupons using the Python Imaging Library (subpackage: ImageDraw). ImageJ (NIH) was used to measure the diameters of the randomly selected fibers.

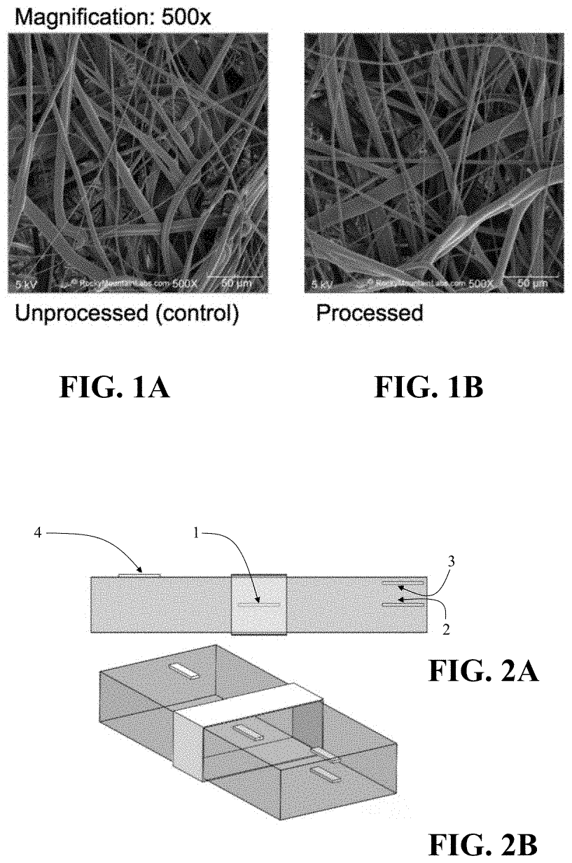

FIG. 1A is an SEM image of a control sample of N95 filter material. FIG. 1B is an SEM image of a sample of N95 filter material after its exposure to ozone according to this Example. The difference in fiber diameters before and after exposure to gaseous ozone was not statistically significant (p=0.1972), indicating that the ozone treatment did not affect the filter fiber diameter or porosity. These results indicate that the treatment with ozone did not compromise the structural integrity of the N95 filter material.

### Example 2: Effect of Ozone Treatment on Spore Strips

This Example illustrates the effect of ozone exposure on Geobacillus stearothermophilus spore strips as bioindicators. Humidity was added to the ozone chamber to increase the relative humidity. High relative humidity together with ozone has been found to increase the kill for virus envelopes, Air and ozone were bubbled through a vessel of water and into a tube that was fed into the chamber. The air became saturated at 100% RR and saturated with ozone molecules. There was still gaseous ozone in the chamber suspended in the air. The addition of aqueous ozone is believed to increase the kill rate.

Geobacillus stearothermophilus spore strips were placed on trays inside the ozone chamber and freely exposed to the environment inside. An ozone concentration of 500 ppm was maintained for 4 hours. The spore strips were then plated on 3M petri film post-treatment and incubated for 72 hours at 25° C. There was no observed change to the bioindicators, and the spores did not appear to be killed.

A second treatment was then performed as above, but this time instead treating the spore strips for 24 hours at a concentration of 500 ppm ozone. The spore strips were plated using the same method as before and incubated for 72 hours at 25° C. The treatment achieved a 3-log reduction—99.9%.

### Example 3: Paper Currency

A strap of 100 paper currency bills was placed on a tray on the racking system in a WillowPure 20 system. Bacillus atropheus spore strips were placed strategically throughout the strap, in positions 1-4 illustrated in FIGS. 2A and 2B, to determine the kill rate within the strap. FIG. 2A illustrates a side view of the strap of bills. FIG. 2B is a perspective view of the strap of bills.

The strap of bills was exposed to an ozone concentration of 450 ppm for 2 hours in the ozone chamber of the system. Table 1 provides the results of the experiment, showing counts of the bacteria at the various positions throughout the strap compared with a control.

TABLE 1

| Position No. | Count |
|---|---|
| 1 | 545,500 |
| 2 | 155,000 |
| 3 | 110,000 |
| 4 | 48,000 |
| Control | 710,000 |

All counts are averages of 2 strips and plates (except control). Spore strips in positions 3 and 4 saw the greatest reduction. There is likely a significant cleaning ability on all outer faces of the bills and about 1-2 inches into the bills from the outer edge.

Embodiments of the disclosure include, for example, those described in the clauses below.

Clause 1. A method for disinfecting an item of personal protective equipment (PPE) contaminated with a virus, wherein the item of PPE is a respiratory protective device or a containment gown, which comprises:

placing the item of PPE in an ozone chamber; and

exposing the item of PPE to gaseous ozone in the ozone chamber at a concentration of at least 50 ppm for at least 10 minutes.

Clause 2. The method of clause 1, wherein the item of PPE is a respiratory protective device.

Clause 3. The method of clause 2, wherein the respiratory protective device is an N95 mask.

Clause 4. The method of clause 1, wherein the item of PPE is a containment gown.

Clause 5. The method of clause 4, wherein the containment gown is made of a material comprising polyethylene, polypropylene, or both.

Clause 6. The method of clause 4, wherein the containment gown is made of a material comprising polyester, cotton, or both.

Clause 7. The method of any one of clauses 1-6, wherein the virus is a respiratory virus.

Clause 8. The method of clause 7, wherein the respiratory virus is SARS-CoV-2.

Clause 9. The method of any one of clauses 1-8, which comprises exposing the item of PPE to gaseous ozone in the ozone chamber at a concentration of at least 100 ppm for at least 10 minutes.

Clause 10. The method of any one of clauses 1-9, which comprises exposing the item of PPE to gaseous ozone in the ozone chamber at a concentration of up to 500 ppm for at least 2 hours.

Clause 11. The method of any one of clauses 1-10, which further comprises exposing the item of PPE to a relative humidity of greater than 95% within the ozone chamber during at least a portion of its exposure to the ozone gas.

Clause 12. A method for disinfecting paper currency contaminated with a virus, which comprises:

placing the paper currency in an ozone chamber; and

exposing the paper currency to gaseous ozone in the ozone chamber at a concentration of at least 50 ppm for at least 10 minutes.

Clause 13. The method of clause 12, wherein the paper currency is in a stack of at least 100 or more individual pieces of paper currency.

Clause 14. The method of any one of clauses 12-13, wherein the virus is a respiratory virus.

Clause 15. The method of clause 14, wherein the respiratory virus is SARS-CoV-2.

Clause 16. The method of any one of clauses 12-15, which comprises exposing the paper currency to gaseous ozone in the ozone chamber at a concentration of at least 100 ppm for at least 10 minutes.

Clause 17. The method of any one of clauses 12-16, which comprises exposing the paper currency to gaseous ozone in the ozone chamber at a concentration of up to 500 ppm for at least 2 hours.

Clause 18. A method for reducing the amount of a virus on an object or material comprising:

containing the object or material in a gaseous ozone chamber;

concentrating gaseous ozone in the chamber and applying the concentrated gaseous ozone to the object or material at a concentration of at least 50 ppm, such as at least 100 ppm, for at least 10 minutes.

Clause 19. The method of clause 18, wherein the object or material is a respiratory protective device or a containment gown.

Clause 20. The method of clause 18, wherein the object or material is paper currency.

I claim:

1. A method for disinfecting an item of personal protective equipment (PPE) contaminated with a virus, wherein the item of PPE is a respiratory protective device or a containment gown, which comprises:
placing the item of PPE in an ozone chamber; and
exposing the item of PPE to gaseous ozone in the ozone chamber at a concentration of at least 50 ppm for at least 10 minutes.

2. The method of claim 1, wherein the item of PPE is a respiratory protective device.

3. The method of claim 2, wherein the respiratory protective device is an N95 mask.

4. The method of claim 1, wherein the item of PPE is a containment gown.

5. The method of claim 4, wherein the containment gown is made of a material comprising polyethylene, polypropylene, or both.

6. The method of claim 4, wherein the containment gown is made of a material comprising polyester, cotton, or both.

7. The method of claim 1, wherein the virus is a respiratory virus.

8. The method of claim 7, wherein the respiratory virus is SARS-CoV-2.

9. The method of claim 1, which comprises exposing the item of PPE to gaseous ozone in the ozone chamber at a concentration of at least 100 ppm for at least 10 minutes.

10. The method of claim 1, which comprises exposing the item of PPE to gaseous ozone in the ozone chamber at a concentration of up to 500 ppm for at least 2 hours.

11. The method of claim 1, which further comprises exposing the item of PPE to a relative humidity of greater than 95% within the ozone chamber during at least a portion of its exposure to the ozone gas.

12. A method for disinfecting paper currency contaminated with a virus, which comprises:
placing the paper currency in an ozone chamber; and
exposing the paper currency to gaseous ozone in the ozone chamber at a concentration of at least 50 ppm for at least 10 minutes.

13. The method of claim 12, wherein the paper currency is in a stack of at least 100 or more individual pieces of paper currency.

14. The method of claim 12, wherein the virus is a respiratory virus.

15. The method of claim 14, wherein the respiratory virus is SARS-CoV-2.

16. The method of claim 12, which comprises exposing the paper currency to gaseous ozone in the ozone chamber at a concentration of at least 100 ppm for at least 10 minutes.

17. The method of claim 12, which comprises exposing the paper currency to gaseous ozone in the ozone chamber at a concentration of up to 500 ppm for at least 2 hours.

18. A method for reducing the amount of a virus on an object or material comprising:
containing the object or material in a gaseous ozone chamber;
concentrating gaseous ozone in the chamber and applying the concentrated gaseous ozone to the object or material at a concentration of at least 50 ppm, such as at least 100 ppm, for at least 10 minutes.

19. The method of claim 18, wherein the object or material is a respiratory protective device or a containment gown.

20. The method of claim 18, wherein the object or material is paper currency.
